(19) United States
(12) Patent Application Publication
(10) Pub. No.: US 2017/0161445 A1
(43) Pub. Date: Jun. 8, 2017

(54) BIOLOGICAL SIGNAL DETECTION METHOD AND ELECTRONIC APPARATUS

(71) Applicant: LeadTone Biotech Inc., New Taipei City (TW)

(72) Inventors: Hsien-Tsai Wu, Hualien County (TW); Kun-Shan Lu, New Taipei City (TW); Wen-Te Lee, New Taipei City (TW)

(21) Appl. No.: 15/197,779

(22) Filed: Jun. 30, 2016

(30) Foreign Application Priority Data
Dec. 2, 2015 (TW) 104140416

(51) Int. Cl. G06F 19/00; A61B 5/0488

(52) U.S. Cl. CPC G06F 19/345; A61B 5/04882

(57) ABSTRACT

A biological signal detection method includes obtaining a bioelectric signal and recording the signal strengths of a plurality of points in the bioelectric signal as a sequence S₁=(Xₙ)_{n∈N} ; converting the sequence S₁ into a sequence

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} < {X}_{i}\end{matrix} , \begin{matrix}1 \leq i \leq n , & 1 \leq j \leq ( n - 1 ) ;\end{matrix}$$

obtaining a group of sub-sequence Dₖ=(Yₖ)_{j≦k≦j+m} from the sequence S₂ with at least one arranging length (m+1), wherein 1≦k≦(n−1−m); counting the number of occurrences of every arrangement in the group of sub-sequence Dₖ and deciding a characteristic arrangement in the arrangements of the group of sub-sequence Dₖ; and comparing the sum of the numbers of occurrences of the characteristic arrangements in the group of sub-sequence Dₖ with a standard threshold, so as to identify a physiological information behind the bioelectric signal. An electronic apparatus for implementing the waveform signal detection is also provided.

### CROSS-REFERENCE TO RELATED APPLICATION

This application claims the priority benefit of Taiwan application serial no. 104140416, filed on Dec. 2, 2015. The entirety of the above-mentioned patent application is hereby incorporated by reference herein and made a part of this specification.

### BACKGROUND OF THE INVENTION

Field of the Invention

The invention relates to a signal processing method and an electronic equipment, and more particularly, to a large-quantity biological signal processing method and an electronic apparatus.

Description of Related Art

Clinical primary bladder neck dysfunction (PBND) is an often neglected cause of lower urinary tract symptoms (LUTS) in males, and males aged 18 to 55 are most susceptible to the disease. The causes of PBND are still unknown today, and the clinical consensus is that PBND is a dysuria caused by the fibrosis of structural bladder neck muscle or fibrous tissue or poor functional bladder neck relaxation.

The conventional treatment for PBND includes using an alpha-blocker first to reduce the resistance of the bladder neck and the prostate. If the drug effects are poor, then the doctor suggests transurethral bladder neck incision (TUI-BN). Although TUI-BN is a relatively common and mature technique and the surgery has a certain efficacy for treating PBND, the symptoms of some patients (roughly 20% to 30%) cannot be alleviated with the surgery. The prior art cannot determine which patients cannot benefit from the surgery beforehand, and patients whose symptoms still cannot be alleviated by surgery selected with other diagnoses do not need to undergo ineffective surgical treatment, in particular TUI-BN surgical treatment.

### SUMMARY OF THE INVENTION

The invention provides a biological signal detection method capable of effectively determining whether the condition of a patient can be improved by surgery beforehand, so as to provide the doctor with assessment of whether an effective surgical treatment is to be performed on the patient.

The invention provides an electronic apparatus capable of testing a subject so as to effectively determine whether the condition of the subject can be improved with surgical treatment.

The biological signal detection method of an embodiment of the invention includes obtaining a bioelectric signal and recording the signal strengths of a plurality of points in the bioelectric signal trending process as a sequence S₁=(Xₙ)_{n∈N}; converting the sequence S₁ into a sequence

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} < {X}_{i}\end{matrix} , \begin{matrix}1 \leq i \leq n , & 1 \leq j \leq ( n - 1 ) ;\end{matrix}$$

obtaining a group of sub-sequence Dₖ=(Yₖ)_{j≦k≦j+m}, from the sequence S₂ with at least one arranging length (m+1), wherein 1≦k≦(n−1−m); counting the number of occurrences of every arrangement in the group of sub-sequence Dₖ and deciding a characteristic arrangement in the arrangements of the group of sub-sequence Dₖ; and comparing the number of occurrences of the characteristic arrangement in the group of sub-sequence Dₖ with a standard threshold, so as to identify a physiological information behind the bioelectric signal.

The electronic apparatus of an embodiment of the invention includes a detector and a processor. The detector is adapted to generate a bioelectric signal. The processor is electrically connected to the detector, wherein the processor is adapted to receive a bioelectric signal and record the signal strengths of a plurality of points in the bioelectric signal as a sequence S₁=(Xₙ)_{n∈N} and convert the sequence S₁ into a sequence

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} < {X}_{i}\end{matrix} , \begin{matrix}1 \leq i \leq n , & 1 \leq j \leq ( n - 1 ) .\end{matrix}$$

The processor obtains a group of sub-sequence Dₖ=(Yₖ)_{j≦k≦j+m} from the sequence S₂ with at least one arranging length (m+1), wherein 1≦k≦(n−1−m); the processor counts the number of occurrences of every arrangement in the group of sub-sequence Dₖ and decides a characteristic arrangement in the arrangements of the group of sub-sequence Dₖ, and compares the number of occurrences of the characteristic arrangement in the group of sub-sequence Dₖ with a standard threshold, so as to identify a physiological information behind the bioelectric signal.

In an embodiment of the invention, the sequence S₂ can be represented as:

$${S}_{2} = ( {Y}_{1} , {Y}_{2} , {Y}_{3} , … , {Y}_{j} ) , \\ \{ \begin{matrix}{Y}_{j} = a , {X}_{i + 1} \geq {X}_{i} \\ {Y}_{j} = b , {X}_{i + 1} < {X}_{i}\end{matrix} , 1 \leq i \leq n , 1 \leq j \leq ( n - 1 ) .$$

In an embodiment of the invention, the sequence S₂ can be represented as:

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} \leq {X}_{i}\end{matrix} , 1 \leq i \leq n , 1 \leq j \leq ( n - 1 ) .$$

In an embodiment of the invention, the sequence S₂ can be represented as:

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = c , {X}_{i + 1} = {X}_{i} \\ {Y}_{j} = b , {X}_{i + 1} < {X}_{i}\end{matrix} , 1 \leq i \leq n , 1 \leq j \leq ( n - 1 ) .$$

In an embodiment of the invention, when the numbers of occurrences of a plurality of characteristic arrangements are obtained with a plurality of different arranging lengths, the numbers of occurrences of the characteristic arrangements are summed and the sum is compared with a standard threshold.

In an embodiment of the invention, the numbers of occurrences of the characteristic arrangements are each multiplied by a weighted value before being summed.

In an embodiment of the invention, the characteristic arrangement is the arrangement having the most number of occurrences in the group of sub-sequence.

In an embodiment of the invention, the waveform signal is an electromyogram signal.

In an embodiment of the invention, the electromyogram signal is a urethral sphincter electromyogram signal of a subject.

In an embodiment of the invention, the electronic apparatus further includes a filter, a signal amplifier, an analog-to-digital converter, and an output. The filter is adapted to filter out a portion of the measured bioelectric signal. The signal amplifier is adapted to amplify the strength of the bioelectric signal. The output is adapted to output a detection signal based on the comparison result of the number of occurrences of the characteristic arrangement in the group of sub-sequence Dₖ with the standard threshold. The filter, the signal amplifier, the analog-to-digital converter, the processor, and the output are electrically connected to one another.

Based on the above, the biological signal detection method in an embodiment of the invention can determine whether surgical treatment is needed for a patient with the arrangement and the number of a portion of the elements in the sequence S₂. The electronic apparatus of an embodiment of the invention can determine whether the symptoms of a patient can be alleviated with surgical treatment by detecting a bioelectric signal beforehand.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic of an electronic apparatus according to an embodiment of the invention.

FIG. 2 is a schematic of a plurality of waveform signals according to an embodiment of the invention.

FIG. 3A is a flow schematic of a biological signal detection method according to an embodiment of the invention.

FIG. 3B is a schematic of a bioelectric signal in the biological signal detection method according to an embodiment of the invention.

FIG. 4 is a flow schematic of a biological signal detection method according to another embodiment of the invention.

### DESCRIPTION OF THE EMBODIMENTS

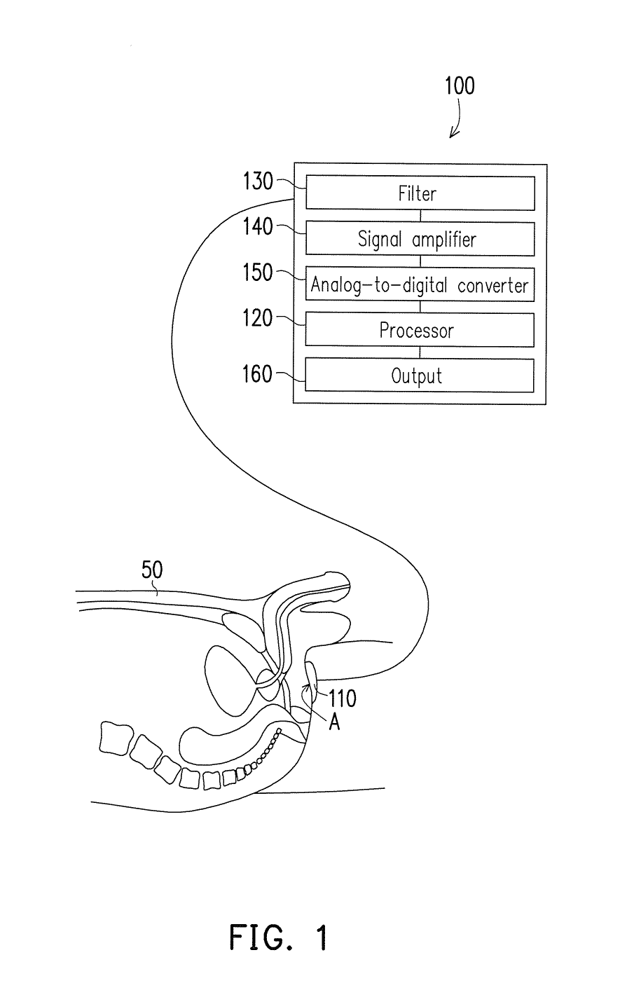

FIG. 1 is a schematic of an electronic apparatus according to an embodiment of the invention. Referring to FIG. 1, in the first embodiment of the invention, an electronic apparatus 100 includes a detector 110 and a processor 120. The detector 110 is adapted to be placed on a test portion A of a laying subject 50 to obtain a waveform signal from the subject 50. The processor 120 is adapted to perform waveform signal detection on the waveform signal.

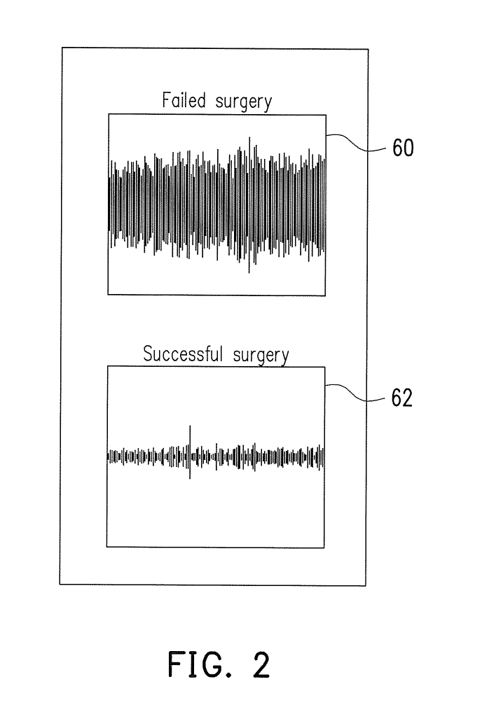

Specifically, FIG. 2 is an actual measurement sampling graph of a plurality of waveform signals according to an embodiment of the invention, in which the test result of patients with failed surgeries shows a waveform signal 60 and the test result of patients with successful surgeries shows a waveform signal 62. The difference between the two waveform signals 60 and 62 cannot be discerned by direct viewing (such as visual inspection). In the following, the biological signal detection method of an embodiment of the invention is exemplarily shown with reference to the waveform signal shown in the figures, and the biological signal detection method of an embodiment of the invention is not limited to the application of the waveform signal.

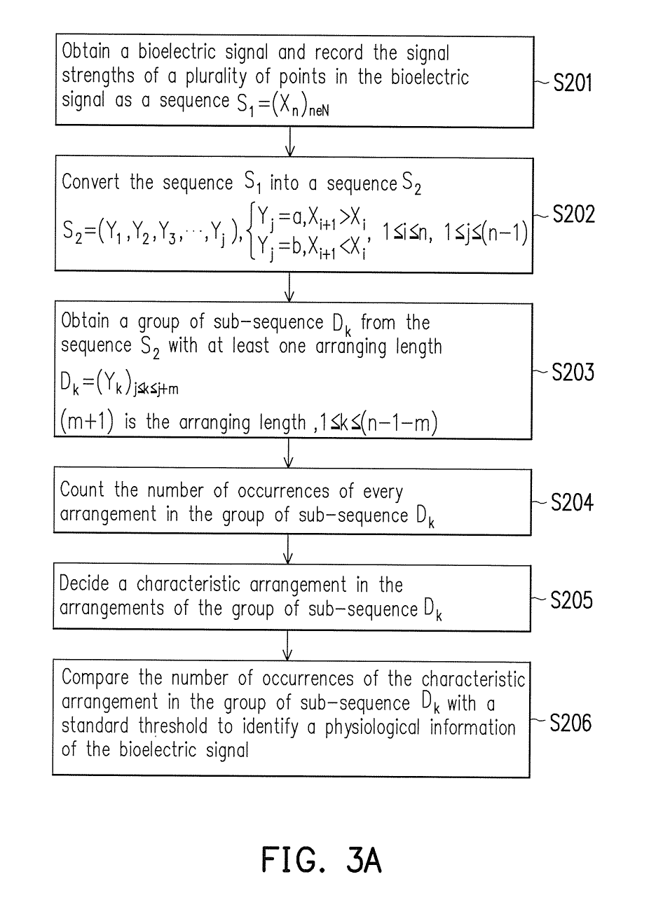

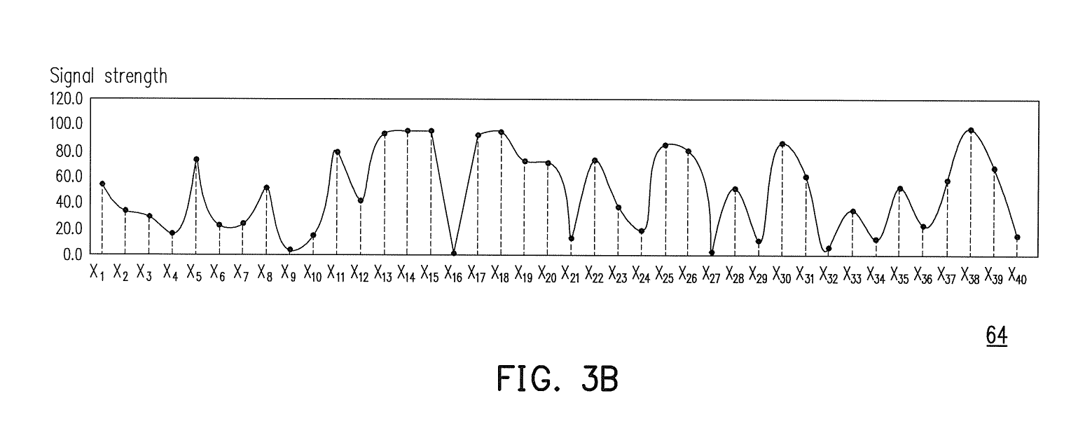

FIG. 3A is a flow schematic of a biological signal detection method according to an embodiment of the invention. Referring to FIG. 3A, in an embodiment of the invention, the biological signal detection method includes obtaining a bioelectric signal and recording the signal strengths of a plurality of points in the bioelectric signal as a sequence S₁=(Xₙ)_{n∈N} (step S201), that is, a bioelectric signal is obtained from, for instance, the above detector 110.

FIG. 3B is a schematic of a bioelectric signal in the biological signal detection method according to an embodiment of the invention. Referring to FIG. 1 and FIG. 3B, specifically, the detector 110 is, for instance, adapted for electromyography detection on the test region A of the subject 50, and the bioelectric signal 64 is, for instance, the electromyography bioelectric signal from a subject. The detector 110 detects the bioelectric signal 64 from the test region A of the subject 50 and sends the bioelectric signal 64 to the processor 120.

Referring to FIG. 3B, the bioelectric signal 64 from the detector 110 is formed by a plurality of data points. For instance, the biological signal detection method of the present embodiment converts the bioelectric signal 64 into a sequence S₁=(X₁, X₂, X₃ . . . X₄₀) having 40 elements at the same intervals, and the numeric element X₁ to the numeric element X₄₀ are arranged into the following numeric sequence S₁ in order:

S₁=(X₁, X₂, X₃, . . . , X₄₀)=(54.1, 34.4, 29.6, 16.6, 73.6, 23.0, 24.2, 51.5, 4.0, 14.9, 79.6, 41.9, 93.6, 96.0, 96.0, 1.0, 92.3, 95.2, 73.1, 71.8, 13.2, 73.5, 37.7, 19.2, 85.0, 80.4, 2.6, 51.6, 11.2, 86.8, 60.8, 5.6, 34.5, 12.7, 52.3, 3.2, 58.2, 97.5, 67.3, 14.9).

Referring to FIG. 3A and FIG. 3B, the biological signal detection method of the present embodiment then converts the sequence S₁ into a sequence

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} < {X}_{i}\end{matrix} , 1 \leq i \leq n , 1 \leq j \leq ( n - 1 ) . \qquad ( step S202 )$$

For instance, in the biological signal detection method of the present embodiment, in the sequence S₂, a is set to 1 and b is set to 0. When adjacent elements (Xᵢ, Xᵢ₊₁) in the sequence S₁ show gradual increase, the i-th element in the sequence S₂ is recorded as 1. When adjacent elements (Xᵢ, Xᵢ₊₁) in the sequence S₁ show gradual decrease, the i-th element in the sequence S₂ is recorded as 0. Therefore, the sequence S₂ can show the gradual increase or gradual decrease situation between every two points of the bioelectric signal using the two elements.

Specifically, the two adjacent elements X₁ and X₂ in the numeric sequence S₁ in the biological signal detection method of the present embodiment are 54.1 and 34.4, wherein the value 34.4 of X₂ is less than the value 54.1 of X₁, and therefore Y₁ in the sequence S₂ can be recorded as 0. On the contrary, the two adjacent elements X₄ and X₅ in S₁ are (16.6, 73.6) in order, wherein the value 73.6 of X₅ is greater than the value 16.6 of X₄, and therefore Y₄ in the sequence S₂ can be recorded as 1.

In the present embodiment, the rule of converting the sequence S₂ further includes when Xᵢ₊₁=Xᵢ, Yⱼ=a (i.e., 1), and therefore the two adjacent elements X₁₄ and X₁₅ in S₁are (96.0, 96.0) in order, wherein the values of X₁₄ and X₁₅ are the same, and Y₁₄ of the sequence S₂ can be recorded as 1.

Therefore, via the above method, the following sequence S₂ formed by the arrangement of the first value (exemplified by 1) and the second value (exemplified by 0) can be obtained:

S₂=(Y₁, Y₂, Y₃, . . . , Y₃₉)=(0, 0, 0, 1, 0, 1, 1, 0, 1, 1, 0, 1, 1, 1, 0, 1, 1, 0, 0, 0, 1, 0, 0, 1, 0, 0, 1, 0, 1, 0, 0, 1, 0, 1, 0, 1, 1, 0, 0).

Specifically, the biological signal detection method of the present embodiment is recorded as the sequence S₂ based on the ups and downs of the bioelectric signal 64 at each time point. That is, the values in the converted sequence S₂ are decided based on whether the bioelectric signal 64 is up or down at each time point. The time points are, for instance, decided by the detection frequency of the detector 110, the bioelectric signal 64 obtains a plurality of values with the detection frequency, and the sequence S₁ is obtained by sampling the values at the same sampling interval or by continuously sampling a plurality of values. However, the invention is not limited thereto.

In other words, since the sequence S₂ of the present embodiment can be represented as

$${S}_{2} = ( {Y}_{1} , {Y}_{2} , {Y}_{3} , … , {Y}_{j} ) , \\ \{ \begin{matrix}{Y}_{j} = a , {X}_{i + 1} \geq {X}_{i} \\ {Y}_{j} = b , {X}_{i + 1} < {X}_{i}\end{matrix} , 1 \leq i \leq n , 1 \leq j \leq ( n - 1 ) , \qquad$$

which corresponds to the ups and downs of the bioelectric signal 64, when two adjacent points in the bioelectric signal 64 are in an increase or a horizontal state (i.e., two corresponding elements of the sequence S₁ are monotonically increasing), the corresponding element of the sequence S₂ is the value a. When two adjacent points in the bioelectric signal are in a decreasing state (i.e., two corresponding elements of the sequence S₁ are in a strictly decreasing state), the corresponding element of the sequence S₂ is the value b, but the invention is not limited thereto. In the biological signal detection method of other embodiments, the sequence S₂ can also be represented as

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} \leq {X}_{i}\end{matrix} , 1 \leq i \leq n , 1 \leq j \leq ( n - 1 ) ,$$

that is, when the values of two adjacent elements in the sequence S₁ are strictly increasing, the corresponding element of the sequence S₂ is defined as the value a, and when the values of two adjacent elements in the sequence S₁ are decreasing in order or are the same, the corresponding element of the sequence S₂ is the value b.

Then, the biological signal detection method of the present embodiment obtains a group of sub-sequence Dₖ=(Yₖ)_{j≦k≦j+m} from the sequence S₂ with at least one arranging length (step S203).

For instance, when the arranging length is 2, the group of sub-sequence includes D₁=(Y₁, Y₂)=(0, 0), D₂=(Y₂, Y₃)=(0, 0), D₃(Y₃, Y₄)=(0, 1), D₄(Y₄, Y₅)=(1, 0) . . . D₃₈=(Y₃₈, Y₃₉)=(0, 0). Since the arranging length (m+1) of the group of sub-sequence is 2 and Dₖ satisfies 1≦k≦(n−1−m), a total of 38 sub-sequences are present.

Then, the biological signal detection method of the present embodiment counts the number of occurrences of every arrangement in each group of sub-sequence Dₖ (step S204).

For instance, when the arranging length is 2, in the group of sub-sequence, the arrangement (0, 0) is repeated 8 times, the arrangement (0, 1) is repeated 12 times, the arrangement (1, 0) is repeated 12 times, and the arrangement is repeated 6 times. Then, the biological signal detection method of the present embodiment, for instance, selects the arrangements (0, 1) and (1, 0) in the group of sub-sequence having the most numbers of occurrences as the characteristic arrangements (step S205), and therefore the highest number of occurrences of the characteristic arrangement of the group of sub-sequence is 12. The characteristic arrangement of the invention is not limited to the arrangement having the most number of occurrences, and in other embodiments, the characteristic arrangement can also be the arrangement having the second most number of occurrences, the least number of occurrences, or the sum of the numbers of occurrences of a portion of the arrangements.

Referring to FIG. 3A, the biological signal processing method of an embodiment of the invention then compares the number of occurrences of the characteristic arrangement with a standard threshold to identify the physiological information of a bioelectric signal (step S206). In other words, the number of occurrences 12 of the arrangements (0, 1) and (1, 0) is, for instance, compared with a standard threshold, and when the number of occurrences is greater than the standard threshold, the complexity of the bioelectric signal is lower than expected, that is, an abnormal state occurs, and the external urethral sphincter tension state of the subject 50 needs to be first lessened, otherwise a TUI-BN surgery will still be insufficient. For instance, when the number of occurrences is less than the standard threshold, the complexity of the bioelectric signal is as expected and the external urethral sphincter of the subject 50 is normal, such that the success rate of improving the condition of the subject 50 with a TUI-BN surgery is higher. In other words, the biological signal method of the present embodiment can suitably quantify the complexity of a bioelectric signal so as to effectively analyze the physiological information behind the bioelectric signal.

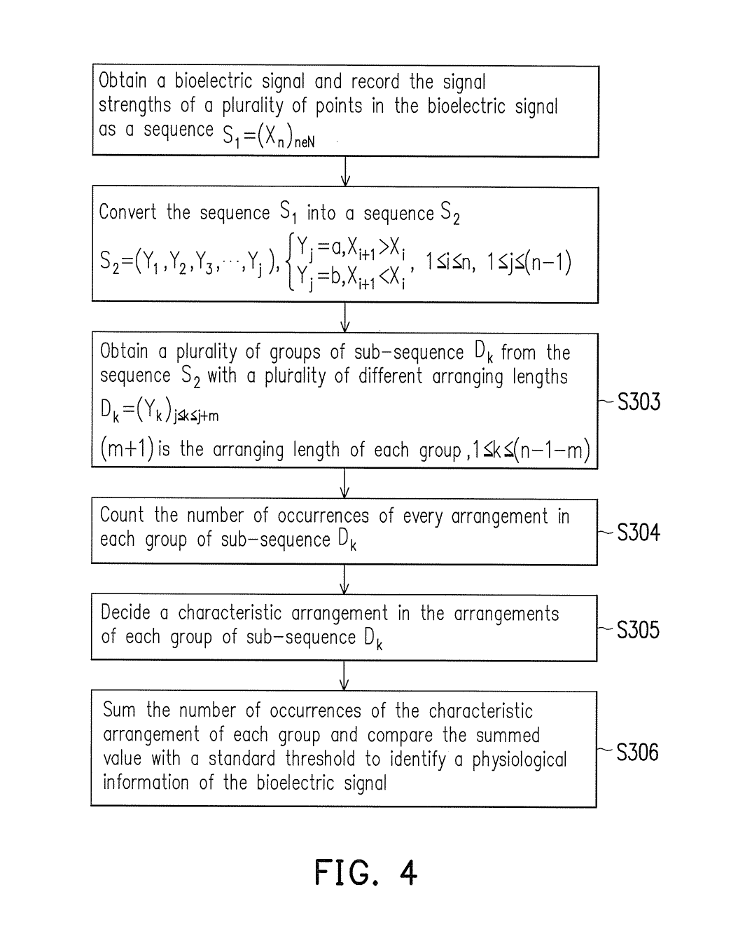

The biological signal processing method of an embodiment of the invention is not limited to the step of obtaining a group of sub-sequence having an arranging length of 2, and in other embodiments, a plurality of groups of sub-sequence can also be obtained from the sequence S₂ having a plurality of different arranging lengths. FIG. 4 is a flow schematic of a biological signal processing method according to another embodiment of the invention. The biological signal processing method of the embodiment shown in FIG. 4 is substantially similar to the biological signal processing method of the embodiment shown in FIG. 3A, and the difference between the two is: the biological signal processing method of the present embodiment obtains a plurality of groups of sub-sequence having a plurality of different arranging lengths after obtaining the sequence S₂ (step S303).

Specifically, in addition to obtaining the group of sub-sequence having an arranging length of 2, the biological signal processing method of the present embodiment also obtains a group of sub-sequence including D₁=(Y₁, Y₂, Y₃)=(0, 0, 0), D₂=(Y₂, Y₃, Y₄)=(0, 0, 1), D₃(Y₃, Y₄, Y₅)=(0, 1, 0), D₄(Y₄, Y₅, Y₆)=(1, 0, 1) . . . D₃₇(Y₃₇, Y₃₈, Y₃₉)=(1, 0, 0) at the same time when the arranging length is 3. Since the arranging length (m+1) of the group of sub-sequence is 3 and Dₖ satisfies 1≦k≦(n−1−m), a total of 37 sub-sequences are present.

In the present embodiment, when the arranging length is 4, a group of sub-sequence including D₁=(Y₁, Y₂, Y₃, Y₄)=(0, 0, 0, 1), D₂=(Y₂, Y₃, Y₄, Y₅)=(0, 0, 1, 0), D₃=(Y₃, Y₄, Y₅, Y₆)=(0, 1, 0, 1), D₄=(Y₄, Y₅, Y₆, Y₇)=(1, 0, 1, 1) . . . D₃₆=(Y₃₆, Y₃₇, Y₃₈, Y₃₉)=(1, 1, 0, 0) is obtained from S₂. Since the arranging length (m+1) of the group of sub-sequence is 4 and Dₖ satisfies 1≦k≦(n−1−m), a total of 36 sub-sequences are present.

Then, in step S304 of the present embodiment, when the arranging length is 3, in the group of sub-sequence, the arrangement (0, 0, 0) is repeated 2 times, the arrangement (0, 0, 1) is repeated 5 times, the arrangement (0, 1, 0) is repeated 7 times, the arrangement (0, 1, 1) is repeated 5 times, the arrangement (1, 0, 0) is repeated 5 times, the arrangement (1, 0, 1) is repeated 7 times, the arrangement (1, 1, 0) is repeated 5 times, and the arrangement (1, 1, 1) is repeated 1 time. For instance, the arrangement having the most number of occurrences in the group of sub-sequence is selected as the characteristic arrangement, and the characteristic arrangements in the sub-sequence having an arranging length of 3 are (0, 1, 0) and (1, 0, 0) having a number of occurrences of 7.

When the arranging length is 4, in the group of sub-sequence, the arrangement (0, 0, 0, 1) is repeated 2 times, the arrangement (0, 0, 1, 0) is repeated 4 times, the arrangement (0, 1, 0, 1) is repeated 4 times, the arrangement (1, 0, 1, 1) is repeated 5 times, the arrangement (0, 1, 1, 0) is repeated 4 times, the arrangement (1, 1, 0, 1) is repeated 3 times, the arrangement (1, 1, 0, 0) is repeated 2 times, the arrangement (0, 1, 1, 1) is repeated 1 time, the arrangement (1, 1, 1, 0) is repeated 1 time, the arrangement (1, 0, 0, 0) is repeated 1 time, the arrangement (0, 1, 0, 0) is repeated 5 times, and the arrangement (1, 0, 0, 1) is repeated 3 times. For instance, the arrangement having the most number of occurrences in the group of sub-sequence is selected as the characteristic arrangement, and the characteristic arrangements in the sub-sequence having an arranging length of 4 are (1, 0, 1, 1) and (0, 1, 0, 0) having a number of occurrences of 5.

It can be known from the above steps that, the number of occurrences of the characteristic arrangements (0, 1) and (1, 0) having an arranging length of 2 is 12, the number of occurrences of the characteristic arrangements (0, 1, 0) and (1, 0, 0) having an arranging length of 3 is 7, and the number of occurrences of the characteristic arrangements (1, 0, 1, 1) and (0, 1, 0, 0) having an arranging length of 4 is 5. Referring to FIG. 4, in step S306 of the biological signal detection method of the present embodiment, the numbers 12, 7, and 5 are summed and then the sum is compared with the standard threshold, and therefore the bioelectric signal can be further quantified. In other words, the biological signal detection method of the present embodiment can observe the ups and downs occurring repeatedly in a bioelectric signal with different lengths, so as to learn whether the bioelectric signal is more complex than the expected normal state.

In an embodiment of the invention, when the bioelectric signal 64 is, for instance, an electromyogram, via the biological signal detection method, the complexity of the bioelectric signal 64 can be quantified, such that physiological information such as the degree of tension of the muscles of a subject can be obtained.

It can be known from the above that, the biological signal detection method of the present embodiment counts the number of occurrences of the order of ups and downs of the bioelectric signal 64 for different arranging lengths to obtain the overall complexity of the bioelectric signal 64. More specifically, via the biological signal detection method, the number of occurrences in the sequence converted by the bioelectric signal 60 shown in FIG. 2 is significantly different from the number of occurrences of the sequence converted by the bioelectric signal 62, such that physiological information in the bioelectric signal 60 and the bioelectric signal 62 can be easily analyzed.

As described above, when the bioelectric signal 64 is, for instance, an electromyogram signal, via the biological signal detection method of the present embodiment, whether the measured muscle is in a tension state can be learned. More specifically, the biological signal 64 is, for instance, the urethral sphincter electromyography signal of the subject, and via the biological signal detection method of the present embodiment, the complexity of the bioelectric signal 64 can be learned, such that whether the contraction function of the sphincter corresponding to the bioelectric signal 64 is normal can be learned. That is, the biological signal detection method of the present embodiment can, for instance, effectively determine whether the condition of a subject can be improved by surgery. Moreover, the electronic apparatus of the present embodiment can execute the biological signal detection method, and therefore whether the symptoms of the subject can be alleviated by surgical treatment can be determined.

Since the biological signal detection method of an embodiment of the invention can suitably quantify the complexity of a bioelectric signal, the bioelectric signal processed thereby is not limited to an electromyogram signal, and the complexity of an electrocardiogram signal or the complexity of other physiological signals can also be determined, so as to analyze the information implied in the physiological information.

In the biological signal detection methods of other embodiments, the sequence S₂ can also be represented as

$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = c , {X}_{i + 1} = {X}_{i} \\ {Y}_{j} = b , {X}_{i + 1} < {X}_{i}\end{matrix} , 1 \leq i \leq n , 1 \leq j \leq ( n - 1 ) ,$$

that is, in the sequence S₂, element c represents the situation in which the values of two continuous elements in the sequence S₁ are the same, element a represents that two adjacent elements in the sequence S₁ are strictly increasing, and element b represents that two adjacent elements in the sequence S₁ are strictly decreasing.

In other embodiments of the invention, in, for instance, step S306, the numbers of occurrences can also each be multiplied by a weighted value before being summed, such that different numbers of occurrences can have different weighted values in the sum. As a result, the biological signal detection method of the present embodiment can be applied in various different bioelectric signals.

In an embodiment of the invention, the electronic apparatus 100 further includes a filter 130 electrically connected to the detector 110. The filter 130 is adapted to filter out a portion of the bioelectric signal from the detector 110. The filter 130 is, for instance, a band pass filter, but the invention is not limited thereto.

The electronic apparatus 100 further includes a signal amplifier 140, an analog-to-digital converter 150, and an output 160 electrically connected to the processor 120. The signal amplifier 140 is adapted to amplify the strength of the bioelectric signal. The analog-to-digital converter 150 is adapted to perform, for instance, the step of converting the bioelectric signal into a numeric sequence. The output 160 is adapted to output a detection signal based on the comparison result of the sum and the standard threshold, and the detection signal is, for instance, the muscle contraction state of the subject. The processor 120 of the present embodiment can be, for instance, a central processing unit (CPU) or a logic circuit, but the invention is not limited thereto.

More specifically, the number of occurrences of the characteristic arrangement or the sum of the numbers of occurrences of a plurality of characteristic arrangements in the above embodiments can also be compared with a plurality of standard thresholds, and the standard thresholds are each categorized to different degrees based on the degree of contraction of the muscle, so as to provide a more comprehensive detection effect to the subject.

Based on the above, the bioelectric signal detection method in an embodiment of the invention can suitably quantify the complexity of the bioelectric signal by counting the numbers of occurrences of the characteristic arrangements or the sum of the numbers of occurrences of a plurality of characteristic arrangements in the sequence S₂, such that comparison can be made with a standard threshold to learn the physiological information in the bioelectric signal. When the bioelectric signal is from a biological electromyogram, the quantized value can determine whether the muscle of a subject is maintained in good condition, so as to learn whether the subject can undergo surgical treatment. The electronic apparatus of an embodiment of the invention can learn the complexity of a bioelectric signal by detecting a bioelectric signal and counting the number of occurrences of the characteristic arrangement therein or the sum of the numbers of occurrences of a plurality of characteristic arrangements so as to determine whether the subject should directly undergo surgical treatment.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A biological signal detection method, comprising:
obtaining a bioelectric signal and recording signal strengths of a plurality of points in the bioelectric signal as a sequence S₁=(Xₙ)_{n∈N};
converting the sequence S₁ into a sequence S₂,
$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} < {X}_{i}\end{matrix} , 1 \leq i \leq n , 1 \leq j \leq ( n - 1 ) ;$$
obtaining a group of sub-sequence Dₖ=(Yₖ)_{j≦k≦j+m} from the sequence S₂ with at least one arranging length, wherein (m+1) is the arranging length, and 1≦k≦(n−1−m);
counting a number of occurrences of every arrangement in the group of sub-sequence Dₖ and deciding a characteristic arrangement in the arrangements of the group of sub-sequence Dₖ; and
comparing a number of occurrences of the characteristic arrangement in the group of sub-sequence Dₖ with a standard threshold to identify a physiological information of the bioelectric signal.

2. The method of claim 1, wherein the sequence is
$${S}_{2} = ( {Y}_{1} , {Y}_{2} , {Y}_{3} , … , {Y}_{j} ) , \\ \{ \begin{matrix}{Y}_{j} = a , {X}_{i + 1} \geq {X}_{i} \\ {Y}_{j} = b , {X}_{i + 1} < {X}_{i}\end{matrix} , 1 \leq i \leq n , 1 \leq j \leq ( n - 1 ) .$$

3. The method of claim 1, wherein the sequence is
$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} \leq {X}_{i}\end{matrix} , 1 \leq i \leq n , 1 \leq j \leq ( n - 1 ) .$$

4. The method of claim 1, wherein the sequence is
$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = c , {X}_{i + 1} = {X}_{i} \\ {Y}_{j} = b , {X}_{i + 1} < {X}_{i}\end{matrix} , 1 \leq i \leq n , 1 \leq j \leq ( n - 1 ) ,$$

5. The method of claim 1, wherein the step of comparing a number of occurrences of the characteristic arrangement in the group of sub-sequence Dₖ with a standard threshold further comprises:
summing the numbers of occurrences of the characteristic arrangements and comparing the sum with the standard threshold when the numbers of occurrences of a plurality of the characteristic arrangements are obtained with a plurality of different arranging lengths.

6. The method of claim 5, further comprising, before the numbers of occurrences of the characteristic arrangements are summed:
multiplying the number of occurrences of each of the characteristic arrangements by a weighted value.

7. The method of claim 1, wherein the characteristic arrangement is an arrangement having a most number of occurrences in the sub-sequences.

8. The method of claim 1, wherein the bioelectric signal is an electromyogram signal.

9. The method of claim 8, wherein the electromyogram signal is a urethral sphincter electromyography signal of a subject.

10. An electronic apparatus, comprising:
a detector adapted to generate a bioelectric signal; and
a processor electrically connected to the detector, wherein the processor is adapted to receive the bioelectric signal and record signal strengths of a plurality of points in the bioelectric signal as a sequence S₁=(Xₙ)_{n∈N} and convert the sequence S₁ into a sequence
$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} < {X}_{i}\end{matrix} , 1 \leq i \leq n , 1 \leq j \leq ( n - 1 ) ,$$
and the processor obtains a group of sub-sequence Dₖ=(Yₖ)_{j≦k≦j+m} from the sequence S₂ with at least one arranging length, wherein (m+1) is the arranging length, and 1≦k≦(n−1−m); the processor counts a number of occurrences of every arrangement in the group of sub-sequence Dₖ and decides a characteristic arrangement in the arrangements of the group of sub-sequence Dₖ, and compares the number of occurrences of the characteristic arrangement in the group of sub-sequence Dₖ with a standard threshold to identify a physiological information of the bioelectric signal.

11. The electronic apparatus of claim 10, wherein when the processor converts the sequence S₂,
$${S}_{2} = ( {Y}_{1} , {Y}_{2} , {Y}_{3} , … , {Y}_{j} ) , \\ \{ \begin{matrix}{Y}_{j} = a , {X}_{i + 1} \geq {X}_{i} \\ {Y}_{j} = b , {X}_{i + 1} < {X}_{i}\end{matrix} , 1 \leq i \leq n , 1 \leq j \leq ( n - 1 ) .$$

12. The electronic apparatus of claim 10, wherein when the processor converts the sequence S₂,
$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = b , {X}_{i + 1} \leq {X}_{i}\end{matrix} , 1 \leq i \leq n , 1 \leq j \leq ( n - 1 ) .$$

13. The electronic apparatus of claim 10, wherein when the processor converts the sequence S₂,
$${S}_{2} = ( {Y}_{1} , {Y}_{2} , {Y}_{3} , … , {Y}_{j} ) , \\ \{ \begin{matrix}{Y}_{j} = a , {X}_{i + 1} > {X}_{i} \\ {Y}_{j} = c , {X}_{i + 1} = {X}_{i} \\ {Y}_{j} = b , {X}_{i + 1} < {X}_{i}\end{matrix} , 1 \leq i \leq n , 1 \leq j \leq ( n - 1 ) .$$

14. The electronic apparatus of claim 10, wherein when the processor obtains numbers of occurrences of a plurality of the characteristic arrangements with a plurality of different arranging lengths, the processor sums the numbers of occurrences of the characteristic arrangements and compares the sum with the standard threshold.

15. The electronic apparatus of claim 14, wherein before summing the numbers of occurrences of the characteristic arrangements, the processor further multiplies the number of occurrences of each of the characteristic arrangements by a weighted value.

16. The electronic apparatus of claim 10, wherein the characteristic arrangement is an arrangement having a most number of occurrences in the sub-sequences.

17. The electronic apparatus of claim 10, wherein the detector is adapted to detect an electromyogram of a subject, and the bioelectric signal is an electromyogram signal.

18. The electronic apparatus of claim 17, wherein the electromyogram signal is a urethral sphincter electromyogram signal of the subject.

19. The electronic apparatus of claim 10, further comprising:
a filter adapted to filter out a portion of the bioelectric signal;
a signal amplifier adapted to amplify a strength of the bioelectric signal;
an analog-to-digital converter; and
an output adapted to output a detection signal based on a comparison result of the number of occurrences of the characteristic arrangement in the group of sub-sequence Dₖ with the standard threshold, wherein the filter, the signal amplifier, the analog-to-digital converter, the processor, and the output are electrically connected to one another.
